(19) United States
(12) Patent Application Publication
(10) Pub. No.: US 2015/0320096 A1
(43) Pub. Date: Nov. 12, 2015

(54) Dietary Supplement

(71) Applicant: MC Corp., San Juan, PR (US)

(72) Inventors: Jorge R. Miranda-Massari, San Juan, PR (US); Michael J. González, Ponce, PR (US); Herbert Massari-Sánchez, San Juan, PR (US)

(73) Assignee: MC Corp, San Juan, PR (US)

(21) Appl. No.: 14/096,048

(22) Filed: Dec. 4, 2013

Related U.S. Application Data
(60) Provisional application No. 61/850,287, filed on Feb. 13, 2013.

(51) Int. Cl. A23L 1/29; A23L 1/30; A23L 1/304; A23L 1/305; A61K 31/51; A61K 31/525; A61K 31/455; A61K 31/197; A61K 31/675; A61K 31/519; A61K 31/14; A61K 31/047; A61K 31/196; A61K 31/4188; A61K 31/714; A61K 31/375; A61K 31/194; A61K 33/30; A61K 33/24; A61K 31/385; A61K 36/54; A61K 38/05; A61K 36/82; A61K 31/122; A61K 9/16; A23L 1/302

(52) U.S. Cl. CPC A23L 1/296; A23L 1/302; A23L 1/30; A23L 1/304; A23L 1/3053; A23L 1/3002; A61K 31/51; A61K 31/525; A61K 31/455; A61K 31/197; A61K 31/675; A61K 31/519; A61K 31/14; A61K 31/047; A61K 31/196; A61K 31/4188; A61K 31/714; A61K 31/375; A61K 31/194; A61K 33/30; A61K 33/24; A61K 31/385; A61K 36/54; A61K 38/05; A61K 36/82; A61K 31/122; A61K 9/16; A23V 2002/00

(57) ABSTRACT

The present dietary supplement improves enzymatic function by supplying the increase demand of nutrients caused by diseases and toxins. The dietary supplement is scientifically designed to balance the Glucose/insulin system and provide energy. The continued use of the present dietary supplement stimulates a metabolic correction by means of providing the necessary cofactors that are lacking or are insufficient in our body, especially when continuously challenged by different stressors.

### RELATED APPLICATIONS

This application claim benefits of U.S. provisional patent application Ser. No. 61/850,287 filed on Feb. 13, 2013.

### STATEMENT REGARDING FEDERALLY SPONSORED RESEARCH AND DEVELOPMENT

N/A

### BACKGROUND OF THE INVENTION

1. Field of the Invention

The present invention relates to unique vitamin, mineral, and herbal supplement for promoting health and metabolic correction. Specifically, the present invention is directed towards a dietary supplement comprising a plurality of compounds from the following group.

2. Discussion of the Background

Diabetes is a disease that affects over 200 million persons globally. This condition is the 5th leading cause of death in the United States. People with diabetes are at significantly higher risk for several chronic conditions which often shortens lifespan, decreases quality of life and requires a substantial economic investment to manage.

Diabetics must undertake lifelong efforts to control their glucose levels and take special care of their health. The scientific literature contains considerable evidence that certain micronutrients and phytonutrients have important roles modifying the metabolism of carbohydrates as well as promoting physiological modulation to improve health.

The highly industrialized nations such as USA rely heavily in synthetic drugs and technology for the management of diseases. This strategy which is based on their current medical standards has created the most expensive, but not necessarily the most cost-effective medicine. In fact, drug induced morbidity and mortality is a very substantial problem for the patient and society. It has been estimated that about 5% of deaths in hospitalized patients adverse drugs events may have caused or contributed to the fatal outcome.

This means that prescribed medications is an important cause mortality, of serious adverse effects leading to health complications and that has significant economic impact. Medications within the medical standards can help achieve some therapeutic benefits, but are limited in the extent of their result by not supplying the metabolic needs, and even worst, causing drug induced nutrient depletion which is one of the causes leading to adverse effects.

In addition death and complications, there is a huge economic impact. Cost of Medication related morbidity and mortality (MM) in ambulatory patients in the USA has increased from $76 billion/yr in 1995 to 177 billion dollars/yr. in 2001. At this rate of increase, the Cost of Medication related MM should surpass $700 billion by 2013.

Further maximum or optimal health requires metabolic harmony. Metabolic Correction is the use of a synergistic combination in sufficient amounts of active nutrient co-factors to improve the biochemical reactions that are fundamental to healthy physiology and to overcome the factors that cause the pathophysiology of a particular condition. In conditions like insulin resistance conducing to pre-diabetes and diabetes, certain enzymes involved in critical reactions are not functioning adequately. By supplying sufficient key co-factors in the most active biochemical forms used in cellular biochemistry, the dysfunctional enzymes can be reactivated which will allow necessary reactions to be reinstated. This way achieving the physiological state needed to improve function. Therefore, there is a need for providing a dietary supplement to control improve the biochemical reactions that are fundamental to healthy physiology, thereby preventing or assisting to overcome the factors that cause the pathophysiology of a particular condition. There is also a need to provide an effective supplement for the treatment of diabetes.

### SUMMARY OF THE INVENTION

The present invention overcomes the limitations of the synthetic drugs by providing a formulation scientifically designed to promote health, balance the Glucose/insulin system and provide energy. The present disclosure defined as a metabolic corrector. The present disclosure, more particularly the present dietary supplement stimulates a metabolic correction by means of providing the necessary cofactors that are lacking or are insufficient in our body, especially when continuously challenged by different stressors.

In accordance with the principles of the present disclosure the dietary supplement optimize enzymatic function, more particularly for a diet high in refined carbohydrates which usually lacks the necessary nutrients needed by our metabolism. When sugar circulates in our blood without being properly utilized and metabolized by the needed target cells (mostly muscle and brain) sugar can be converted to fat. The dietary supplement improves health by providing the necessary cofactors that may enable the body to improve carbohydrate metabolism, reducing carbohydrate/sugar cravings, in addition to helping diminish its conversion to fat, as well as favoring its utilization for energy producing reactions.

Another objective of the invention is to provide a comprehensive formula designed to improve or correct carbohydrate metabolism.

Another objective of the invention is to provide a comprehensive formula designed to promote healthy physiology of nerve.

Another objective of the invention is to provide a comprehensive formula designed to support and promote healthy micro-vascular tissues.

The invention itself, both as to its configuration and its mode of operation will be best understood, and additional objects and advantages thereof will become apparent, by the following detailed description of a preferred embodiment taken in conjunction with the accompanying drawing.

The Applicant hereby asserts, that the disclosure of the present application may include more than one invention, and, in the event that there is more than one invention, that these inventions may be patentable and non-obvious one with respect to the other.

Further, the purpose of the accompanying abstract is to enable the U.S. Patent and Trademark Office and the public generally, and especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

### BRIEF DESCRIPTION OF THE DRAWINGS

The accompanying drawings, which are incorporated herein, constitute part of the specifications and illustrate the preferred embodiment of the invention.

FIG. 1 shows a table with glucose patient's results after using the preferred embodiment of the invention in accordance with the principles of the present disclosure.

FIG. 2 shows a table with A1C patient's results after using the preferred embodiment of the invention in accordance with the principles of the present disclosure.

### DESCRIPTION OF THE PREFERRED EMBODIMENT

The present invention focuses upon a new and unique dietary supplement formulation scientifically designed to balance the Glucose/insulin system and provide energy. The continued use of the present dietary supplement induces a metabolic correction by means of providing the necessary nutrients that are lacking or are insufficient in our body when continuously challenged.

Formulation comprising:

| | | |
|---|---|---|
| Thiamin | 10 | mg |
| Riboflavin | 10 | mg |
| Niacin | 10 | mg |
| Pantothenic acid | 10 | mg |
| Pyridoxal phosphate | 10 | mg |
| L-metylfolate | 800 | mcg |
| Choline | 5 | mg |
| Inositol | 5 | mg |
| PABA | 1 | mg |
| Biotin | 1 | mg |
| Methylcobalamin | 50 | mcg |
| Vitamin C | 250 | mg |
| Magnesium citrate | 25 | mg |
| Zinc picolinate | 10 | mg |
| Chromium polynicotinate | 200 | mcg |
| Vanadium sulphate | 500 | mcg |

### Proprietary Blend Comprises:

| | | |
|---|---|---|
| R-Alpha Lipoic acid | 150 | mg |
| Acetyl L-carnitine | 100 | mg |
| Cinnamon extract | 50 | mg |
| L-Carnosine | 50 | mg |
| Green Tea Extract | 50 | mg, |
| Coenzyme Q10 | 10 | mg |

### Formulation:

Metabolic Corrector—

Synergistic MicroNutrient Combination in sufficient amount of the most active chemical forms to enhance the enzymatic activity and metabolic reactions of the cell. These include biochemical physiological pathways such as: glycolysis, Krebs cycle, Electron Transport System, formation of nitric oxide and catabolism of homocysteine and other intermediate metabolites.

In addition to the synergistic combination of active co-factors to improve and correct metabolism, further improvements in physiology are possible by the use of specific botanical compounds known to have biological activity in enzymes, membrane, receptors and reduction-oxidation reactions among many others. This proposed combination of metabolic correctors and physiologic modulators can produce results that can transform a dysfunctional organ or system and reverse tissue damage into achieve to restore normal physiology and achieve necessary tissue repair or regeneration. Since some of the ingredients of this proposed formulation are native molecules necessary for the metabolic processes, they are well tolerated and the body has multiple uses (pleiotropic) for them as well as well effectively developed transformation and elimination systems, thus explaining their high level of tolerability.

The use of products of natural origin facilitates the production of relatively inexpensive formulation which is an important consideration given that the treatment of conditions of insulin-sugar system dysfunction is becoming an economic burden to individuals and of our modern societies. We propose the formulation of a cost-effective Metabolic Corrector with co-factors and botanical products for the control of blood glucose and the promotion healthy nerve tissues based on scientific and clinical evidence.

### Ingredient Action:

1—B-Complex

(Thiamin, Riboflavin, Niacin, Pantothenic acid, Choline, Inositol, PABA, Biotin)

B vitamins are crucial to the metabolism of carbohydrates, fats and proteins and in the production of energy. In relation to carbohydrate management, Biotin is especially important for improving insulin sensitivity and the activity of glucokinase, the enzyme that starts the use of glucose by the liver. Diabetics have low concentrations of this enzyme. Research shows that supplementation with Biotin improves blood glucose control in both Type I and Type II diabetics. Biotin is also helpful for treating diabetic neuropathy, which is damage to the nerves that causes numbness, burning sensation and pain.

2—L-metylfolate (5-MTHF)

L-metylfolate is the primary biologically active isomer of folate and the form of folate in circulation. It is also the form which is transported across membranes into peripheral tissues, particularly across the blood brain barrier. There is a genetic defect or polymorphism of this enzyme in about 50% of the general population that affects a person's ability to fully convert folic acid to L-methylfolate. Reduced levels of L-methylfolate will reduce methylation and increase homocysteine which in turn may increased the risk of myocardial infarction, stroke, depression, migraine, birth defects, diabetic nephropathy and memory loss.

3—Methylcobalamin

Vitamin B12 may have a strong role to play when treating diabetic neuropathy. The presence of vitamin B12 is necessary for the correct functioning of nerve cells and therefore taking it as a supplement may help to reduce nerve damage. In extreme cases, the extra effect of intramuscular B12 may be necessary.

Methylcobalamin is one biologically active form of vitamin B₁₂. Methylcobalamin is the principal form of circulating vitamin B12, hence the form which is transported into peripheral tissue. Methylcobalamin is absorbed by a specific intestinal mechanism which uses an intrinsic factor and by a diffusion process in which approximately 1% of the ingested dose is absorbed. Cyanocobalamin and hydroxycobalamin are forms of the vitamin that require conversion to Methylcobalamin via the intermediate glutathionyl-B12. As we age our bodies reduce the absorption of B12 and folate. Methylcobalamin is a cofactor of the enzyme methionine synthase, which functions to transfer methyl groups for the regeneration of methionine from homocysteine. By increasing folate levels and maintaining normal levels of B12, we decrease homocysteine and vascular risks including cognitive impairment. Elevated levels of homocysteine can be a metabolic indication of decreased levels of the methylcobalamin form of vitamin B12. Oral administration of methylcobalamin resulted in subjective improvement of burning sensations, numbness, loss of sensation, and muscle cramps. An improvement in reflexes, vibration sense, lower motor neuron weakness and sensitivity to pain was also observed.

4—Pyridoxal Phosphate

Neuropathy can be caused by high blood sugar levels and may be also associated with deficiency of vitamin B6, (pyridoxine). Pyridoxine may be able to improve glucose tolerance, particularly for sufferers from gestational diabetes or impaired glucose tolerance caused by the birth control pill. Vitamin B6 also has a strong role to play in the prevention of diabetes-related complications.

Pyridoxal-5′-phosphate (PLP) is the active form of vitamin B6 and is used as the prosthetic group for many enzymes. Pyridoxine, the parent compound of PLP and the most frequently used form of vitamin B6, requires reduction and phosphorylation before becoming biologically active. Pyridoxal Phosphate is necessary for the activation of glycine in the initial stages of heme production. A direct correlation has been found between carpal tunnel syndrome (CTS) and a deficiency in P5P, and its use has been reported to be beneficial in CTS. B6 nutritional status has a significant and selective modulatory impact on the production of both serotonin and GABA, the neurotransmitters that control depression, pain perception and anxiety. P5P is a cofactor in the synthesis of these neurotransmitters. High levels of plasma homocysteine are considered an independent risk factor for atherosclerotic disease and venous thrombosis. Homocysteine, an intermediate in methionine metabolism, can be re-methylated to methionine, or be channeled down the trans-sulfuration pathway to cysteine, which requires two P5P-dependent enzymes: cystathionine synthase and cystathionase.

5—Vitamin C

Vitamin C helps prevent glycosylation of proteins. These substances are associated with diabetic complications in the eyes, kidneys and circulatory system as well as with increased levels of free radicals. Vitamin C is one of the safest of all supplements even at high levels. Heart disease is a common complication of diabetes. As high blood pressure and arterial stiffness are both risk factors for heart disease, the results of this study suggest that vitamin C supplementation can reduce the chance that a person with diabetes will develop this complication. Previous research has shown that diabetics have higher requirements for vitamin C than healthy people. Vitamin C, which has a chemical structure similar to that of the common sugar glucose, appears to compete with glucose for entry into cells. When the blood glucose level is elevated, as in diabetes, more vitamin C than usual is needed in order for the vitamin to perform its functions in the body. In experimental animals, vitamin C deficiency causes hardening of the arteries (atherosclerosis). Research has suggested that vitamin C might also help prevent other consequences of diabetes, such as damage to the eyes and nerves. Vitamin C inhibits all three of the biochemical reactions that are believed to contribute to the development of these complications: (1) the production of oxygen derived free radicals, (2) the accumulation of sorbitol within cells, and (3) the tissue-damaging reaction called glycosylation. Type 1 diabetics generally have low vitamin C levels. By increasing the amount of vitamin c in the bloodstream, the amount of sorbitol may be lowered. Sorbitol is a harmful sugar when it accumulates, and its presence may lead to increased risk of diabetic complications such as retinopathy, neuropathy and kidney damage. In the case of type 2 diabetics, vitamin c may play a role in improving glucose tolerance.

6—Magnesium Citrate

While this mineral is not directly implicated in the mechanisms of diabetes, it helps to protect patients from complications of the disease. Magnesium acts to relax smooth muscle tissue, including those lining the arteries and therefore helps lower blood pressure and reduces the risks of heart attacks and strokes. Deficiencies in magnesium are also linked to diabetic retinopathy; diabetics with the lowest levels of magnesium had the worst retinopathy.

7—Zinc Picolinate

Zinc plays a key role in the regulation of insulin production by pancreatic tissues and glucose utilization by muscles and fat cells. The abilities to synthesize and secrete insulin and use glucose are impaired in the zinc deficient state. Intestinal zinc absorption rates and plasma zinc levels in diabetic patients are reduced. Zinc is involved in the regulation of insulin receptor-initiated signal transduction mechanisms and insulin receptor synthesis.

8—Chromium Polynicotinate

Chromium is an essential trace mineral. Chromium levels in the body tend to decline with age, which may be one factor affecting older people's risk of developing Type II diabetes. Chromium is known to enhance the action of insulin, a hormone critical to the metabolism and storage of carbohydrate, fat, and protein in the body. In 1957, a compound in brewers' yeast was found to prevent an age-related decline in the ability of rats to maintain normal levels of sugar (glucose) in their blood. Chromium was identified as the active ingredient in the glucose tolerance factor. Chromium also appears to be directly involved in carbohydrate, fat, and protein metabolism. People with diabetes take chromium in an effort to improve their blood glucose control. Chromium supplementation has been researched for its effect on glucose control in people with diabetes. Chromium Polynicotinate Chromium polynicotinate, also known as niacin-bound chromium, may be the preferred form to use because it binds to niacin (Vitamin B-3), this provides a biologically active form of chromium that makes it easier for the body to absorb.

9—Vanadium Sulphate

Vanadium supplements produce a slight increase in insulin sensitivity and may therefore allow diabetic patients to decrease the amount of insulin that they need to keep their blood sugar levels under control. Studies in both animals and humans have proved the association between vanadium levels and normal blood glucose. Research indicates that this mineral acts similarly to insulin in transporting glucose into the cells and is therefore valuable for both Type I and Type II diabetics.

10—Alpha-Lipoic Acid

(ALA, lipoic acid, thioctic acid) is an antioxidant, a substance that protects against free radical damage. Alpha lipoic acid is called a universal antioxidant. It is a very versatile molecule. It is able to work in both fatty and aqueous parts of the body, which means it can work within and outside the cell. Other powerful antioxidants are only soluble in fat, i.e. Vitamin E, or in water such as Vitamin C. Vitamin E is usually found in the fatty portion of cell membranes while vitamin C is found in the interior of cells and in the blood. ALA is the only known antioxidant that can easily get into the brain, so it is not surprising that it is being used in the treatment of Alzheimer. ALA is particularly protective of the DNA and mitochondria. Glucose and fats produce in the mitochondria adenosine triphosphate (ATP), the chemical that the body uses as energy.

The body needs the enzymes involved in ATP production to work properly and these need ALA. It helps by activating these enzymes, which is why it is referred to as a coenzyme. If this energy conversion does not work properly, less glucose is burned so it builds up in the bloodstream, leading to hypoglycemia. People with type 2 diabetes take ALA supplements to lower blood glucose levels by improving the body's ability to use insulin; for Type 2 diabetics getting the glucose into the cell is of primary importance as insulin resistance hampers that process. ALA appears to be an insulin mimetic and works alongside insulin to make the process happen. ALA is also used to prevent and treat diabetic neuropathy. ALA has been researched for its effect on insulin sensitivity, glucose metabolism and diabetic neuropathy. It has been shown to improve glucose utilization in many tissues but especially in muscle tissue, apparently by causing an increase in the number of glut-4 transporters on the outside of the muscle cells, sometimes by as much as 50%. This is particularly important because muscle is responsible for the greatest uptake of glucose after a meal. So basically ALA increases the glucose stored in muscle by channeling more of the glucose from the bloodstream to the muscle instead of to the adipose (fat) tissue. Diabetics also suffer from increased glycation, which is where glucose tends to bind with proteins and damages them. ALA helps reduce this glycation and rids the body if the free radicals.

11-Acetyl L-Carnitine (ALCAR)

Carnitine is required by the body in order to correctly use body fat in the production of energy. Diabetics using carnitine respond well by reducing high levels of fat in the bloodstream (cholesterol and triglycerides). Carnitine helps to break down fatty acids in the body and binds acyl residues. For these reasons, it may be useful to prevent diabetic ketoacidosis. ALCAR promotes peripheral nerve regeneration and has been shown to have analgesic effects in patients with HIV-related or chemotherapeutic origin and DPN.

12—Cinnamon

Studies into the effects of cinnamon on Type 2 diabetics has shown that taking cinnamon can lead to a reduction in average fasting glucose levels (between 20% and 30%) and a reduction in cholesterol levels of around 10% to 25%. Cinnamon appears to be useful to help control blood glucose levels and reducing cholesterol, the dual effect may reduce the risk of stroke and heart disease. Even type 1 diabetics can benefit from cinnamon intake, reducing their high blood sugar levels.

13—Carnosine

The amino acid carnosine, is an antioxidant that stabilizes and protects cell membranes. Carnosine prevents glycosylation, wherein protein molecules bind to glucose molecules in the body to form nonfunctioning structures. There is strong evidence that the proper dose of carnosine is the safest and most effective method of inhibiting glycosylation, which may help to prevent age-related conditions such as muscle atrophy, eye problems, and neurological degeneration.

12—Green Tea Extract

Polyphenols-antioxidants found in green tea-are being studied for effects on vascular health (including blood pressure) and on the body's ability to use insulin. Laboratory studies suggest that EGCG, a polyphenol found in green tea, may protect against cardiovascular disease and have a beneficial effect on insulin activity and glucose control. Green tea is safe for most adults.

11—Coenzyme Q10

Coenzyme Q10 benefits energy metabolism and ATP production in patients with diabetes. In patients with diabetes, an ATP producing enzyme that requires coenzyme Q10 to function was measured. The amount and activity of the enzyme were significantly lower in diabetics than in non-diabetics. Some oral drugs used to treat diabetes were shown to cause a further decrease in coenzyme Q10 in addition to that caused by the diabetes. Dietary supplementation with coenzyme Q10 in patients with diabetes significantly increased plasma coenzyme Q10 by at least 3-fold. Studies of dietary supplementation with of coenzyme Q10 have been shown to significantly reduce blood glucose levels and lower hemoglobin AlC (HbAlC), a measure of blood sugar control over 2 to 3 months. Furthermore, coenzyme Q10 supplementation has been reported to reduce insulin requirements in patients with diabetes. Many patients with diabetes also have elevated blood pressure, which is a significant risk factor for development of the long-term complications of diabetes described above. Coenzyme Q₁₀ significantly lowered blood pressure in patients with diabetes.

The compound may include additional botanicals. The followings are:

Mormodica charantia—Fruit, 1 gm Bid

Mormodica charantia Is a climbing vine that grows in the wet tropical countries such as South America, India and the Caribbean. The extracts of this plant have been found to contain mormocharin and mormodicin which are thought to possess insulin like chemical structure and properties. The hypoglycemic effect is believed to be due to depression of key gluconeogenic enzymes or the increase in the concentration of glucose transporters and stimulation of glucose uptake in skeletal muscle cells. In addition, preservation of the pancreatic islet β cell and has been related to a significant increase in insulin secretory activity.
1. Insulin secretagoge effect.
2. Stimulation of peripheral and skeletal muscle glucose utilization.
3. Inhibition of intestinal glucose uptake.
4. Inhibition of hexokinase activity.
5. Suppression of key gluconeogenic enzymes.
6. Stimulation of key enzyme if HMP pathway.
7. Preservation of islet beta cells and their function.

Gymena Sylvestre.

Leaf extract. 500 mg per day in clinical trial or liquid form (extract). 25 to 75 ml per week. Gymnema Sylvestre is a herb widely distributed in India, Australia, many Asian countries and tropical Africa. The use leaf extract has been found to produce hypoglycemic effects which are thought to be the effect of Gymnemic acid. Some of the effects that have been found include: 1) Induces regeneration of islet cells, 2) augmentation of insulin secretion, 3) inhibition of glucose absorption from intestine, 4) increased utilization of glucose by it increasing the activities of enzymes responsible for utilization of glucose by insulin-dependent pathways.

Patients with diabetes type 2 in controlled clinical trials supplemented with Gymnema had improvement in fasting glucose, glycated hemoglobin and other metabolic variables.

Cissus sicyoides.

Aqueous extract of leaves. Cissus sicyoides is a vine common in Brazil and other tropical areas and islands. The aqueous extract from this plant was found to have hypoglycemic and anti-lipidemic effects. The mode of action of the aqueous extract of Cissus sicyoides does not resemble insulin of sulfonylureas, but may be similar to biguanides which inhibit gluco-neogenesis. Aqueous extracts of leaves of C. sicyoides have been found to contain antioxidants that may be useful in the deterrence of diabetic complications related to oxidative stress. Administration of its aqueous extracts promotes substantial decreases in glucose levels after 60 days of administration in animal models. The gastro-protective effect of the methanolic extract of Cissus sicyoides in the rodent model was confirmed and was associated with an increase of the defense mechanism of the gastrointestinal mucosa such as nitric oxide (NO) and sulphidryl (SH) groups.

Manufacturing Process
1. Obtain material designated previously at the formulation.
2. The obtained materials are weighed according to the present disclosure, more particularly the formulation in a weighing area. The weight of each one of the ingredients for this formula is verified by a Quality Control Personnel.
3. The weighed ingredients are mixed in stainless steel mixers until a homogeneous mixture is obtained. This process is carefully monitored by a Quality Control Personnel.
4. The final mixture is emptied into food-grade plastic bags (liners) within coated plastic containers. This mixture is kept in clean rooms at appropriate temperature and humidity until it is sent to the encapsulating areas.
5. The mixture is encapsulated in clean rooms especially designed for this procedure.
6. After the encapsulation process, the capsules are polished and inspected, to eliminate any defective product.
7. The capsules are then bottled and labeled automatically in Dark Amber PET (polyethylene terephthalate) bottles, closed with white heat induction caps.
8. Finally the bottles are packed inside a double carton box.

### EXAMPLE

Illustrated is the effect of the composition according to the present disclosure. At least 4 different patients were treated for a period not minor to 4 months to a top of 12 months. The preferred doses do not exceed two capsules per day, however other doses might be recommended by physicians.

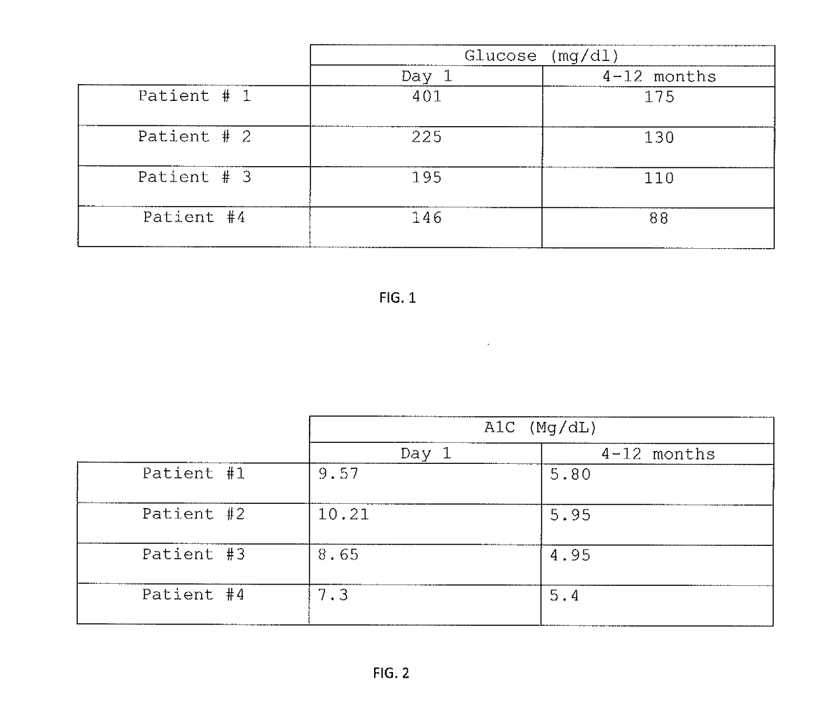

FIG. 1 clearly shows that the glucose level decreases after several month of using the present disclosure compound. Further, hemoglobin A1C decreases, as shown in FIG. 2, with the use of the present compound.

It is apparent from the results that the dietary supplement composition in accordance with the principles of the present disclosure balances the Glucose and hemoglobin A1C by reducing the abnormal level presented by patients.

All of the patents, patent applications, and publications recited herein, and in the Declaration attached hereto, if any, are hereby incorporated by reference as if set forth in their entirety herein. All, or substantially all, the components disclosed in such patents may be used in the embodiments of the present invention, as well as equivalents thereof. The details in the patents, patent applications, and publications incorporated by reference herein may be considered to be incorporable at applicant's option, into the claims during prosecution as further limitations in the claims to patently distinguish any amended claims from any applied prior art.

What is claimed is:

1. A nutritional supplement, comprising a source of active material, wherein said active material comprises a source of thiamin, a source of riboflavin, a source of niacin, a source of pantothenic acid, a source of pyridoxal phosphate, a source of L-metylfolate, a source of choline, a source of Inositol, a source of PABA, a source of biotin, a source of methylcobalamin, a source of vitamin C, a source of magnesium citrate, a source of zinc, a source of picolinate, a source of chromium polynicotinate, a source of vanadium sulphate, a source of R-Alpha lipoic acid, a source of acetyl L-carnitine, a source of cinnamon extract, a source of L-Carnosine, a source of green tea extract and a source of Coenzyme Q10.

2. The nutritional supplement of claim 1, wherein said source of active material is blended into a homogeneous compound.

3. The nutritional supplement of claim 2, wherein the said source of active material is encapsulated.

4. The nutritional supplement of claim 3, wherein the said source of active material is encapsulated in an inactive source material.

5. The nutritional supplement of claim 1, wherein said source of active material comprises a source of gymnema sylvestre, mormodica charantia and cissus sicyoides.

6. A method for providing nutrition to a patient comprising:
enterally administering to the patient an effective amount of a composition comprising a source of active material, wherein said active material comprises a source of thiamin, a source of riboflavin, a source of niacin, a source of pantothenic acid, a source of pyridoxal phosphate, a source of L-metylfolate, a source of choline, a source of Inositol, a source of PABA, a source of biotin, a source of methylcobalamin, a source of vitamin C, a source of magnesium citrate, a source of zinc, a source of picolinate, a source of chromium polynicotinate, a source of vanadium sulphate, a source of R-Alpha lipoic acid, a source of acetyl L-carnitine, a source of cinnamon extract, a source of L-Carnosine, a source of green tea extract and a source of Coenzyme Q10.

7. The method for providing nutrition to a patient of claim 6, wherein said source of active material is blended into a homogeneous compound.

8. The method for providing nutrition to a patient of claim 7, wherein the said source of active material is encapsulated.

9. The method for providing nutrition to a patient of claim 8, wherein the said source of active material is encapsulated in an inactive source material.

10. The method for providing nutrition to a patient of claim 6, wherein said source of active material comprises a source of Gymnema Sylvestre, Mormodica charantia and Cissus sicyoides.
